(19) United States
(12) Patent Application Publication
(10) Pub. No.: US 2020/0107753 A1
(43) Pub. Date: Apr. 9, 2020

(54) Systems and Methods for Utilizing Gravity to Determine Subject-Specific Information

(71) Applicant: UDP Labs, Inc., Los Gatos, CA (US)

(72) Inventors: Steven Jay Young, Los Gatos, CA (US); Carl Hewitt, San Jose, CA (US); Jonathan Olson, San Jose, CA (US); Alan Luckow, Ben Lomond, CA (US); Robert Dobkin, Monte Sereno, CA (US)

(21) Appl. No.: 16/551,087

(22) Filed: Aug. 26, 2019

Related U.S. Application Data
(60) Provisional application No. 62/804,623, filed on Feb. 12, 2019.
(60) Provisional application No. 62/742,613, filed on Oct. 8, 2018.

(51) Int. Cl. A61B 5/11; A61B 5/00; G08B 21/22

(52) U.S. Cl. CPC A61B 5/1115; A61B 5/1102; A61B 2560/0223; G08B 21/22; A61B 5/6891

(57) ABSTRACT

A system for measuring data specific to a subject using gravity comprises a substrate on which a subject lies, the substrate having multiple legs extending from the substrate to a floor to support the substrate, and load sensor assemblies. Each load sensor assembly is associated with a respective leg and comprises a cap configured to receive a load from the substrate, a base configured to provide contact with the floor, the base and cap configured to fit together to maintain alignment of the cap to the base while allowing vertical movement of the cap, a load cell between the base and the cap, one of the base and cap configured to translate the load to the load cell and a printed circuit board that processes and outputs data from the load cell, wherein a combination of all load sensor assemblies receive an entire load to which the substrate is subjected.

### CROSS-REFERENCE TO RELATED APPLICATION(S)

This application claims priority to and the benefit of U.S. Provisional Application Patent Ser. No. 62/742,613, filed Oct. 8, 2018 and U.S. Provisional Application Patent Ser. No. 62/804,623, filed Feb. 12, 2019, the entire disclosure of which is hereby incorporated by reference.

### TECHNICAL FIELD

This disclosure relates to systems and methods for sensing biometrics and other subject-specific information of one or more subjects using multiple sensors that are not in contact with the subjects.

### BACKGROUND

Sensors have been used to detect heart rate, respiration and presence of a single subject using ballistocardiography and the sensing of body movements using noncontact methods, but are often not accurate at least due to their inability to adequately distinguish external sources of vibration and distinguish between multiple subjects. In addition, the nature and limitations of various sensing mechanisms make it difficult or impossible to accurately determine a subject's biometrics, presence, weight, location and position on a bed due to factors such as air pressure variations or the inability to detect static signals.

### SUMMARY

Disclosed herein are implementations of systems for measuring data specific to a subject using gravity. One such system comprises a substrate on which a subject lies, the substrate having multiple legs extending from the substrate to a floor to support the substrate, and load sensor assemblies. Each load sensor assembly is associated with a respective leg and comprises a cap configured to receive a load from the substrate, a base configured to provide contact with the floor, the base and cap configured to fit together to maintain alignment of the cap to the base while allowing vertical movement of the cap, a load cell between the base and the cap, one of the base and cap configured to translate the load to the load cell and a printed circuit board that processes and outputs data from the load cell, wherein a combination of all load sensor assemblies receive an entire load to which the substrate is subjected.

Another embodiment of a system for measuring data specific to a subject using gravity comprises a substrate on which a subject rests, the substrate having multiple legs extending from the substrate to a floor to support the substrate, at least two load sensor assemblies, each load sensor assembly associated with a respective leg configured to measure a static load and changes in load on the substrate through the leg, a controller and communication means from each of the at least two load sensor assemblies to the controller, wherein the controller processes output from each of the at least two load sensor assemblies.

### BRIEF DESCRIPTION OF THE DRAWINGS

The disclosure is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity.

FIG. 1 is schematic of a system for measuring data specific to a subject using gravity.

FIGS. 2A and 2B are schematics of a load sensor assembly as disclosed herein.

FIGS. 3A and 3B are embodiments of load sensor assemblies as disclosed herein.

FIGS. 4 and 5 are embodiments of systems for measuring data specific to a subject using gravity.

FIGS. 6A and 6B are schematics of a system for measuring data specific to a subject using gravity using a floor mat.

FIG. 7 is a schematic of a system for measuring data specific to a subject using gravity incorporated into a floor.

FIG. 8 is an exploded view of another embodiment of a load sensor assembly as disclosed herein.

FIG. 9 is a schematic of a leg of a substrate incorporating an accelerometer sensor assembly.

FIGS. 10A and 10B are schematics of a system incorporating an optical vibration sensor as disclosed herein.

FIGS. 11A and 11B are schematics of a knife edge sensor assembly as disclosed herein.

FIGS. 12A and 12B are schematics of an optical encoder sensor assembly as disclosed herein.

FIGS. 12C-12G are embodiments of templates used with the optical encoder sensor assembly.

FIGS. 13A-13C are schematics of a polarized sensor assembly as disclosed herein.

FIG. 14 is a schematic of a fiber optics power source for the systems disclosed herein.

FIG. 15A is a plan view of a system for measuring data specific to one subject using gravity.

FIG. 15B is a plan view of a system for measuring data specific to two subjects using gravity.

FIG. 16 is a diagram of signal adding to increase signal strength.

FIG. 17A is a schematic of a system for measuring data specific to a subject using gravity and canceling out external noise.

FIG. 17B is a diagram of signal cancellation to remove external noise.

FIG. 18A represents different loads on the load sensor assemblies based on a sleeping position.

FIG. 18B represents the loads on the load sensor assemblies based on another sleeping position.

FIG. 19 is a schematic illustrating a substrate having legs that lower the substrate to accommodate a subject exiting the substrate.

FIG. 20 represents the loads on the load sensor assemblies based on yet another sleeping position.

### DETAILED DESCRIPTION

Disclosed herein are implementations of systems and methods employing gravity and motion to determine biometric parameters and other person-specific information for single or multiple subjects at rest and in motion on one or multiple substrates. The systems and methods use multiple sensors to sense a single subject's or multiple subjects' body motions against the force of gravity on a substrate, including beds, furniture or other objects, and transforms those motions into macro and micro signals. Those signals are further processed and uniquely combined to generate the person-specific data, including information that can be used to further enhance the ability of the sensors to obtain accurate readings. The sensors are connected either with a wire, wirelessly or optically to a host computer or processor which may be on the internet and running artificial intelligence software. The signals from the sensors can be analyzed locally with a locally present processor or the data can be networked by wire or other means to another computer and remote storage that can process and analyze the real-time and/or historical data.

The sensors are designed to be placed under, or be built into a substrate, such as a bed, couch, chair, exam table, floor, etc. The sensors can be configured for any type of surface depending on the application. Additional sensors can be added to augment the system, including light sensors, temperature sensors, vibration sensors, motion sensors, infrared sensors and sound sensors as non-limiting examples. Each of these sensors can be used to improve accuracy of the overall data as well as provide actions that can be taken based on the data collected. Example actions might be: turning on a light when a subject exits a bed, adjusting the room temperature based on a biometric status, alerting emergency responders based on a biometric status, sending an alert to another alert based system such as: Alexa, Google Home or Ski for further action.

The data collected by the sensors can be collected for a particular subject for a period of time, or indefinitely, and can be collected in any location, such as at home, at work, in a hospital, nursing home or other medical facility. A limited period of time may be a doctor's visit to assess weight and biometric data or can be for a hospital stay, to determine when a patient needs to be rolled to avoid bed sores, to monitor if the patient might exit the bed without assistance, and to monitor cardiac signals for atrial fibrillation patterns. Messages can be sent to family and caregivers and/or reports can be generated for doctors.

The data collected by the sensors can be collected and analyzed for much longer periods of time, such as years or decades, when the sensors are incorporated into a subject's personal or animal's residential bed. The sensors and associated systems and methods can be transferred from one substrate to another to continue to collect data from a particular subject, such as when a new bed frame is purchased for a residence or retrofitted into an existing bed or furniture.

The highly sensitive, custom designed sensors detect wave patterns of vibration, pressure, force, weight, presence and motion. These signals are then processed using proprietary algorithms which can separate out and track individual source measurements from multiple people, animals or other mobile or immobile objects while on the same substrate.

These measurements are returned in real-time as well as tracked over time. Nothing is attached to the subject. The sensors can be electrically or optically wired to a power source or operate on batteries or use wireless power transfer mechanisms. The sensors and the local processor can power down to zero or a low power state to save battery life when the substrate is not supporting a subject. In addition, the system may power up or turn on after subject presence is detected automatically.

The system is configured based on the number of sensors. Because the system relies on the force of gravity to determine weight, sensors are required at each point where an object bears weight on the ground. For other biometric signals fewer sensors may be sufficient. For example, a bed with four wheels or legs may require a minimum of four sensors, a larger bed with five or six legs may require five for six sensors, a chair with four legs would may require sensors on each leg, etc. The number of sensors is determined by the needed application. The unique advantage of multiple sensors provides the ability to map and correlate a subject's weight, position and bio signals. This is a clear advantage in separating out a patient's individual signals from any other signals as well as combining signals uniquely to augment the signals for a specific biosignal.

The system can be designed to configure itself automatically based on the number of sensors determined on a periodic or event-based procedure. A standard configuration would be four sensors per single bed with four legs to eight leg sensors for a multiple person bed. The system would automatically reconfigure for more or less sensors. Multiple sensors provide the ability to map and correlate a subject's weight, position and bio signals. This is necessary to separate multiple subjects' individual signals.

Some examples of the types of information that the disclosed systems and methods provide are dynamic center of mass and center of signal locations, accurate bed exit prediction (timing and location of bed exit), the ability to differentiate between two or more bodies on a bed, supine/side analysis, movement vectors for multiple subjects and other objects or animals on the bed, presence, motion, position, direction and rate of movement, respiration rate, respiration condition, heart rate, heart condition, beat to beat variation, instantaneous weight and weight trends, and medical conditions such as heart arrhythmia, sleep apnea, snoring, restless leg, etc. By leveraging multiple sensors that detect the z-axis and other axes of the force vector of gravity, and by discriminating and tracking the center of mass or center of signal of multiple people as they enter and move on a substrate, not only can the disclosed systems and methods determine presence, motion and cardiac and respiratory signals for multiple people, but they can enhance the signals of a single person or multiple people on the substrate by applying the knowledge of location to the signal received. Secondary processing can also be used to identify multiple people on the same substrate, to provide individual sets of metrics for them, and to enhance the accuracy and strength of signals for a single person or multiple people. For example, the system can discriminate between signals from an animal jumping on a bed, another person sitting on the bed, or another person lying in bed, situations that would otherwise render the signal data mixed. Accuracy is increased by processing signals differently by evaluating how to combine or subtract signal components from each sensor for a particular subject.

Additional sensor types can be used to augment the signal, such as light sensors, temperature sensors, accelerometers, vibration sensors, motion sensors and sound sensors.

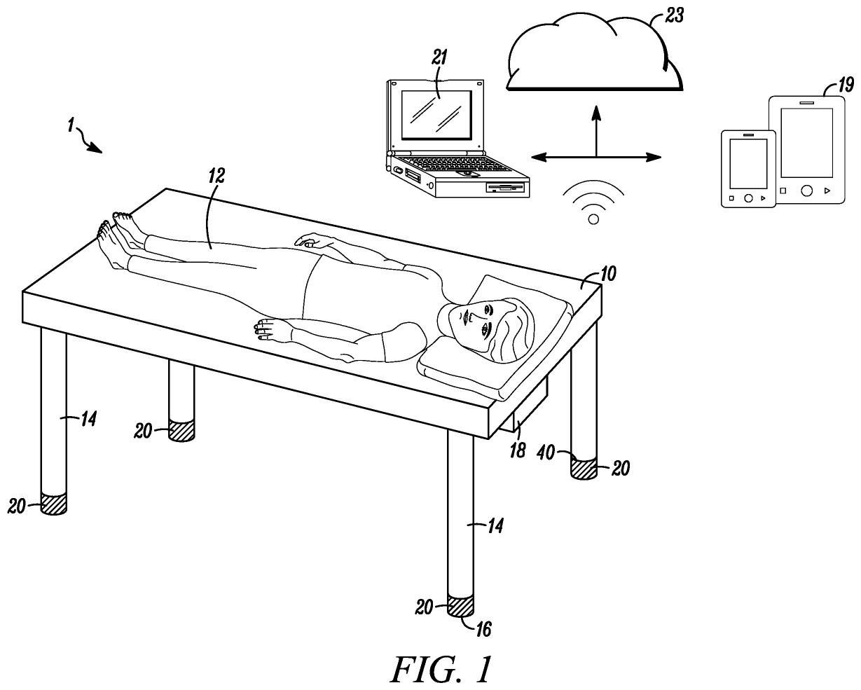

FIG. 1 illustrates a system 1 for measuring data specific to a subject using gravity. The system 1 can comprise a substrate 10 on which a subject 12 can lie, the substrate 10 having multiple legs 14 extending from the substrate 10 to a floor 16 to support the substrate 10. Multiple load sensor assemblies 20 can be used, each load sensor assembly 20 associated with a respective leg 14 of the substrate 10. Any point in which a load is transferred from the substrate 10 to the floor 16 should have an intervening load sensor assembly 20.

As illustrated in FIG. 1, a local controller 18 can be wired or wirelessly connected to the load sensor assemblies 20 and collects and processes the signals from the load sensor assemblies 20. The controller 18 can be attached to the frame of the substrate so that it is hidden from view, can be under the substrate or can be positioned anywhere a wireless transmission can be received from the load sensor assemblies 20 if transmission is wireless. The controller 18 can be programmed to control other devices based on the processed data as discussed below, the control of other devices also being wired or wireless. Alternatively, or in addition to, an off-site controller 21 or a cloud-based network 23 can collect the signals directly from the load sensor assemblies 20 for processing or can collect raw or processed data from the local controller 18. For example, the local controller 18 may process the data in real time and control other local devices as disclosed herein, while the data is also sent to the off-site controller 21 that collects and stores the data over time. The controller 18 or 21 may transmit the processed data off-site for use by downstream third parties such as medical professionals, fitness trainers, family members, etc. The controller 18 or 21 can be tied to infrastructure that assists in collecting, analyzing, publishing, distributing, storing, machine learning, etc. Design of real-time data stream processing has been developed in an event-based form using an actor model of programming. This enables a producer/consumer model for algorithm components that provides a number of advantages over more traditional architectures. For example, it enables reuse and rapid prototyping of processing and algorithm modules. As another example, it enables computation to be location-independent (i.e., on a single device, combined with one or more additional devices or servers, on a server only, etc.)

The long-term collected data can be used in both a medical and home setting to learn and predict patterns of sleep, illness, etc. for a subject. As algorithms are continually developed, the long-term data can be reevaluated to learn more about the subject. Sleep patterns, weight gains and losses, changes in heart beat and respiration can together or individually indicate many different ailments. Alternatively, patterns of subjects who develop a particular ailment can be studied to see if there is a potential link between any of the specific patterns and the ailment.

The data can also be sent live from the local controller 18 or the off-site controller 21 to a connected device 19, which can be wirelessly connected for wired. The connected device 19 can be, as examples, a mobile phone or home computer. Devices can subscribe to the signal, thereby becoming a connected device 19.

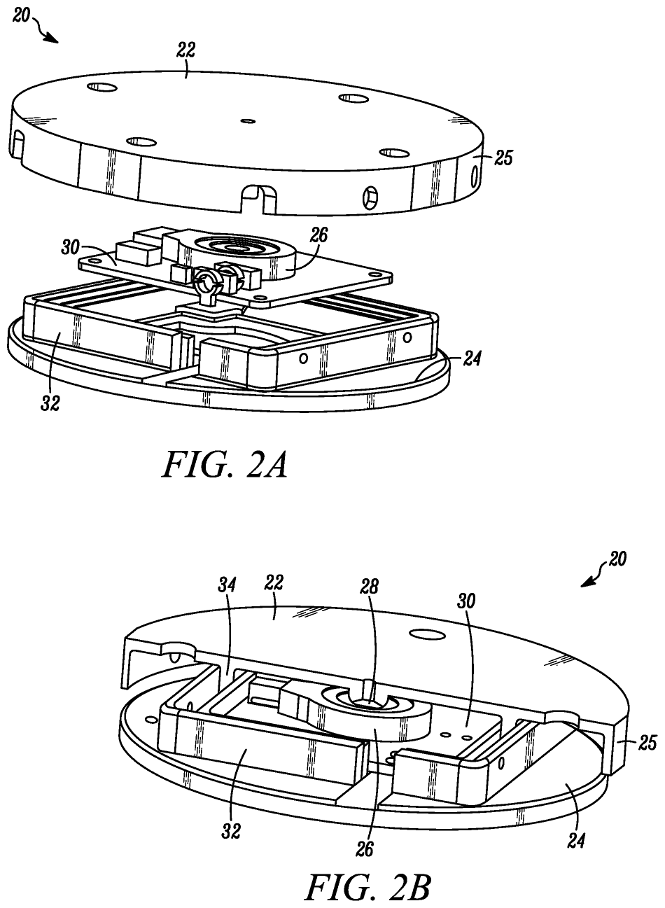

As illustrated in FIGS. 2A and 2B, each load sensor assembly 20 comprises a cap 22 configured to receive a load from the substrate 10 and a base 24 configured to provide contact with “ground”, or the floor 16, the base 24 and cap 22 configured to fit together to maintain alignment of the cap 22 to the base 24 while allowing vertical movement of the cap 22. The base's contact with the floor 16 can be direct or indirect, such as through the leg 14 of the substrate 10. A load cell 26 is positioned between the base 24 and the cap 22, and one of the base 24 and cap 22 is configured to translate the load to the load cell 26. For example, the load cell 26 may be secured to the base 24 and the cap 22 may translate the load directly or indirectly, through a cell contact surface 28, to the load cell 26. Alternatively, the load cell 26 may be secured to the cap 22, and the base 24 may directly, or indirectly through a different circuit contact surface, transfer the load to the load cell 26. The load cell 26 can also be a strain sensor. A printed circuit board 30 between the base 24 and the cap 22 processes and outputs data from the load cell 26 to one or both of the local controller 18 and the off-site controller 21. The base 24 provides containment features to trap the walls of the cap from moving horizontally while allowing movement of the cap 22 vertically to transfer the load. The containment feature can be a double walled portion 32 on the base 24 in which a corresponding single wall 34 on the cap 22 is received.

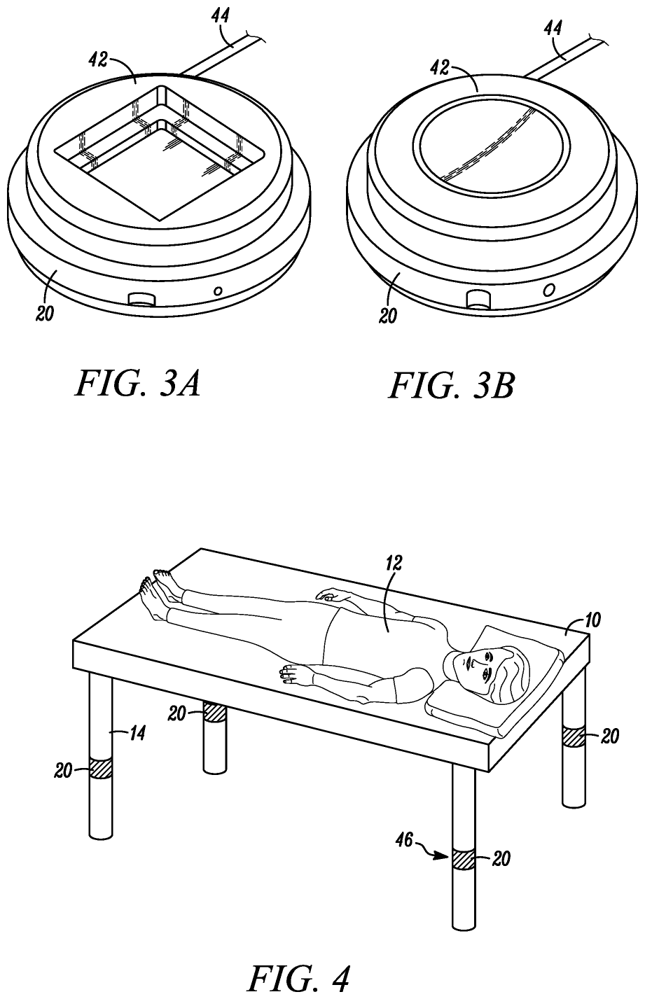

The load sensor assemblies 20 can be incorporated into the top, bottom or any portion of the legs 14 of the substrate 10. For aesthetic reasons, the cap 22 can have a perimeter 25 sized and shaped to be identical to a perimeter of a leg 14, with the base 24 fitting within the cap 22. Alternatively, the base 24 can have a perimeter sized and shaped to be identical to the perimeter of the leg 14, with the cap 22 fitting within the base 24. As illustrated in FIG. 1, the load sensor assemblies 20 are on the bottom 40 of the leg 14. The load sensor assemblies 20 can be physically attached to the bottom 40 of the leg 14 so that they move when the substrate 10 and legs 14 are moved. Alternatively, the load sensor assemblies 20 can be configured with a leg receiver 42, as illustrated in FIGS. 3A and 3B. The leg receivers 42 can be shaped to best contain the bottom 40 of the leg 14 while receiving the entire load born through the leg 14. The leg receivers 42 can be integral with the cap 22 or can be attached to the cap 22. The load sensor assemblies 20 as shown in FIGS. 3A and 3B with wires 44 that can be either power to the load sensor assemblies 20 or can be data transmitted from the load sensor assemblies 20. The wires 44 can be hidden along the leg 14 and frame of the substrate 10 for aesthetics.

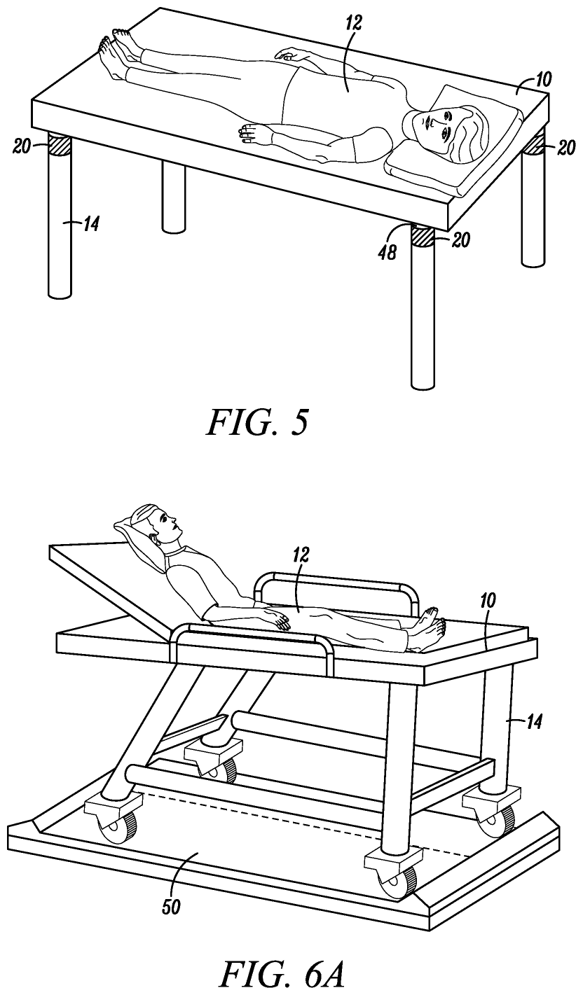

FIG. 4 illustrates the load sensor assemblies 20 inline in the middle 46 of each leg 14 while FIG. 5 illustrates the load sensor assemblies 20 at the top 48 of each leg 14. The load sensor assemblies 20 can be incorporated between the substrate frame and the legs 14, for example. The load sensor assemblies 20 can be placed directly on top of the leg 14 or can be fitted into a hollow of the leg, so long as the entire load from the substrate 10 to the floor 16 in that location goes through the sensor assembly 20.

The load sensor assemblies 20 can also be incorporated into the castors of wheels, i.e., between the legs 14 and the castors of substrates that are on wheels, such as hospital beds.

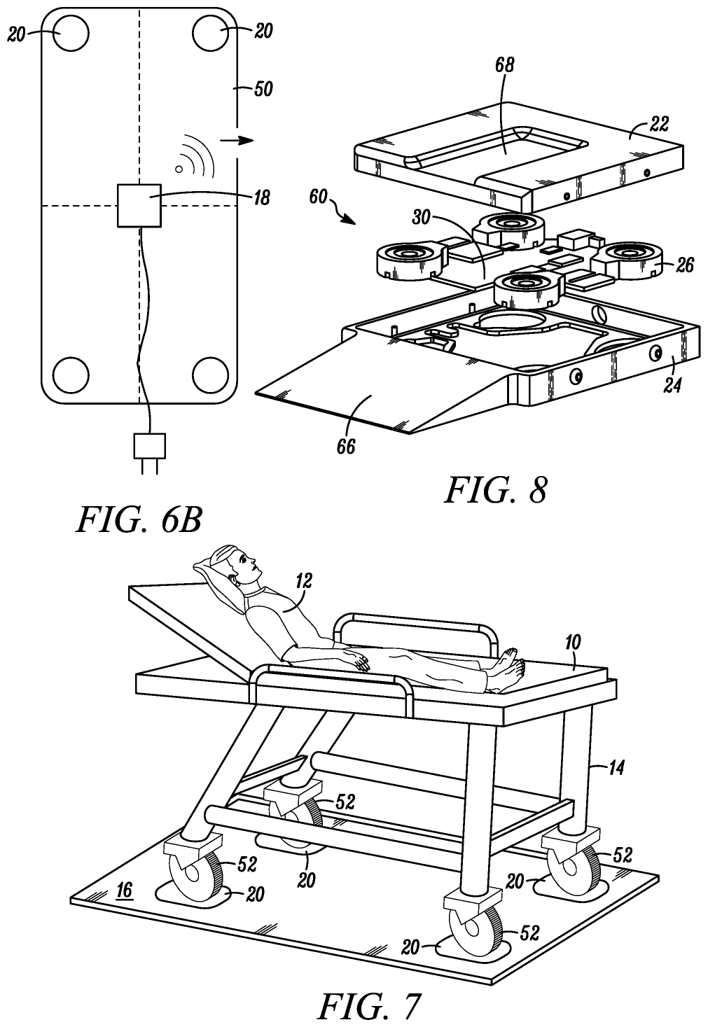

As illustrated in FIGS. 6A and 6B, the load sensor assemblies 20 can be located in floor mats 50 that are used to create bays onto which beds on wheels or castors can be rolled and positioned when use of the load sensor assemblies 20 is desired. The floor mat 50 is sized to have an area at least as large as an area defined by the legs 14 of the substrate 10. The base 24 of the load sensor assemblies 20 can be in direct contact with the floor 16 when incorporated into the mat 50 or can have some mat 50 intervening between it and the floor 16. The bed can be rolled onto the mat 50 and positioned such that legs 14 are on the load sensor assemblies 20. The mats 50 can be positioned on the floor of a medical facility, for example, to create “bays” in which a bed can be rolled into when use of the load sensor assemblies 20 is desired for a specific patient. Each mat 50 can have a corresponding local controller 18 that can communicate with connected devices 19 and/or other computers. The load sensor assemblies 20 in the mat 50 can be wired to the local controller 18 through the matt 50 so the wires are hidden. The local controller 18 can also provide power to the sensor assemblies 20.

The load sensor assemblies can be arranged in the floor 16 on which the substrate 10 sits or on which the substrate 10 is positioned, as illustrated in FIG. 7. For example, the load sensor assemblies 20 can be placed in an opening in the floor 16 so that the cap 22 is flush with the floor 16. The substrate 10 may have legs 14 with wheels 52 that can be rolled over the load sensor assemblies 20 so that the legs 14 are directly on the assemblies. The load sensor assemblies 20 can be permanently positioned in the floor to create “bays” in which a bed can be rolled into when use of the load sensor assemblies 20 is desired for a specific patient.

To provide for a larger footprint for use with heavy loads, particularly in hospital and other medical facilities, each load sensor assembly 60 can have multiple load cells 26 positioned on the base 24 with the cap 22 configured with a cell contact surface 28 configured to translate the load through the respective leg 14 equally to each of the multiple load cells 26, as illustrated in FIG. 8. An array of load cells 26 is spaced around a center of the assembly 60 such that when the leg 14 is positioned on the assembly 60, the load is spread equally to the load cells 26. The circuit board 30 is positioned in the base 24. The large footprint load sensor assemblies 60 can be placed directly on the floor 16 and can each further include a ramp 66 to allow for rolling a substrate 10 such as a hospital bed on wheels up the ramps 66 until the legs 14 are correctly positioned. The cap 22 can also have an indentation 68 sized to fit a wheel to prevent the wheel from rolling off of the large footprint load sensor assembly 60 and to help with proper positioning of the respective loads.

In addition, or alternative to the load sensor assemblies described, other types of sensors can be used. Other types of sensors can be used in a combination with load cells to enhance the accuracy and quality of data, in cases where higher resolution is needed, or when the application of load cells is not possible or practical based on the characteristics of the substrate. For example, when it is not practical to place more than four legs at the corners of a bed, yet signal acquisition is desired near the middle of the bed. Additional sensors can also be substituted for load cells in cases where the additional information provided by load cells is not required.

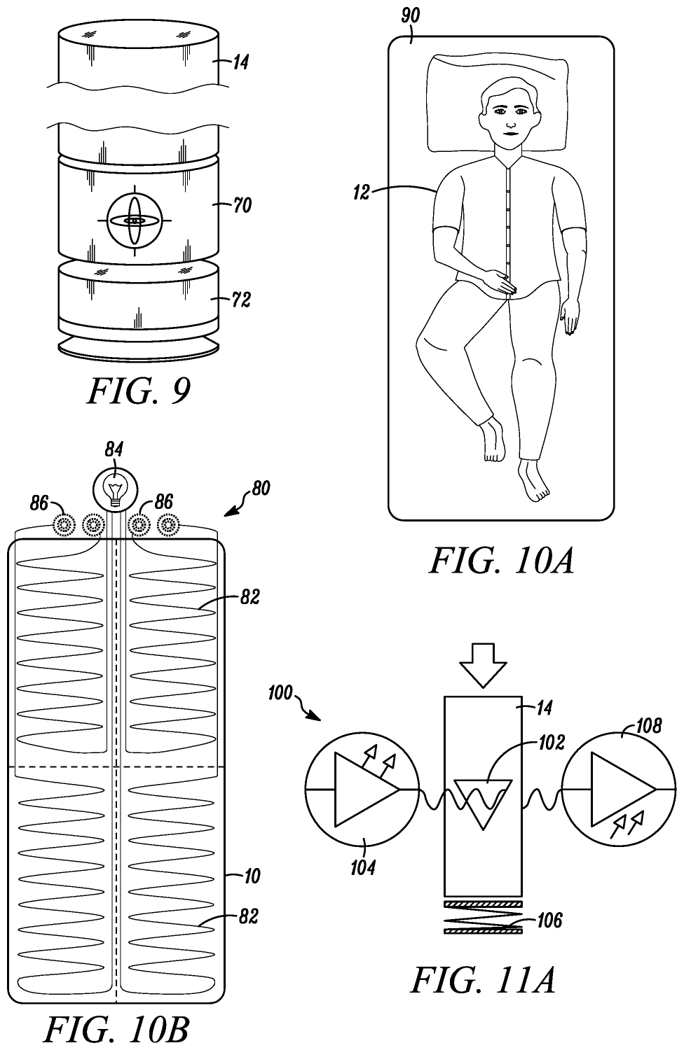

One or more accelerometers 70 can be used with the system 1. Accelerometers measure acceleration forces, which can be static, like the continuous force of gravity, or may be dynamic, sensing movement or vibrations. This acceleration is caused by tilt with respect to the earth. The substrate “tilts” due to blood flow, physical movement and respiration of the subject. The output from the accelerometers can be analyzed in the same way that the output from the load sensor assemblies can be used. The accelerometer(s) can be placed anywhere in or on the legs as described with respect to the load sensor assemblies 20 or can be placed anywhere on the substrate 10 itself. However, when the accelerometer 70 is used in a leg 14 of the substrate 10, flex material 72 is positioned under the accelerometer 70 as illustrated in FIG. 9. The flex material 72 amplifies the signal, allowing for very subtle transfer of motion and providing a higher strength movement signal.

One or more piezoelectric sensors can be used with the system. The piezoelectric sensor uses the piezoelectric effect to measure changes in pressure, acceleration, temperature, strain or force by converting them to an electrical charge. Similar algorithms can be applied to the output from the piezoelectric sensors to obtain data pertaining to the subject or subjects on the substrate. Piezoelectric sensors are typically sheet-like, such that the piezoelectric sensors can be placed directly under the substrate or can be placed between the substrate and the subjects, as examples.

FIGS. 10A and 10B illustrate the use of an optical vibration sensor system 80 which uses optical fibers 82. In an optical fiber 82, light travels through the core even if the fiber is twisted. Some of the light signal degrades within the fiber 82, often due to impurities in the glass but also due to movement of the fiber. The extent that the signal degrades depends upon the purity of the glass and the wavelength of the transmitted light. This degradation is used to calculate biometric data. As illustrated, four optical vibration sensor systems 80 are used with each covering a quarter of the area of the substrate 10. A light source 84 provides light to the optical fiber 82 and the signal from each optical fiber 82 is transmitted to a respective sensor 86. The length of the fiber and the way in which the optical fiber is laid down is known, and the algorithms used to manipulate the sensor data is based in part on these parameters. The way in which the optical fiber is laid down in FIG. 10B is provided as a non-limiting example. FIG. 10A illustrates a mattress 90 laid over the optical vibration sensor systems 80, which are positioned on the substrate 10.

In addition to or alternative to one or more of the load sensor assemblies 20 previously described, a knife edge sensor assembly 100 can be used, as illustrated in FIGS. 11A and 11B. The knife edge sensor assembly 100 includes a knife edge opening 102 at which light 104 is directed. For example, the knife edge opening 102 may be formed in the leg 14 of the substrate 10. As another example, the knife edge opening 102 may be formed in the body of a sensor that is positioned in the leg 14. The sensor, or leg, is positioned on a spring-like device 106, or alternatively, a flexible substrate that is sufficiently flexible to allow for movement of the knife edge opening 102. As pressure is placed on the leg 14, as illustrated in FIG. 11A, the knife edge opening 102 moves and some portion of light signal is transmitted through the knife edge opening 102. The amount of light that is transmitted through the opening 102 equates to a load or motion on the substrate. As illustrated in FIG. 11B, the light signal may be completely interrupted when there is no load on the substrate. The light 104 is transmitted through the opening 102 to a photodiode 108 that measures the amount of light transmitted. From the data from the photodiode 108, presence, movement and weight thresholds can be measured. For example, presence can be determined based on a change from no light transmitted to any amount of light transmitted. Weight thresholds or ranges can be determined from a change from no light being transmitted to a specific amount of light being transmitted, wherein each amount of light corresponds to a weight on the substrate. Movement such as turning over is determined from a change in the amount of light being transmitted. Even movement such as breathing can be measured based on very small changes in the amount of light detected and the frequency of those changes.

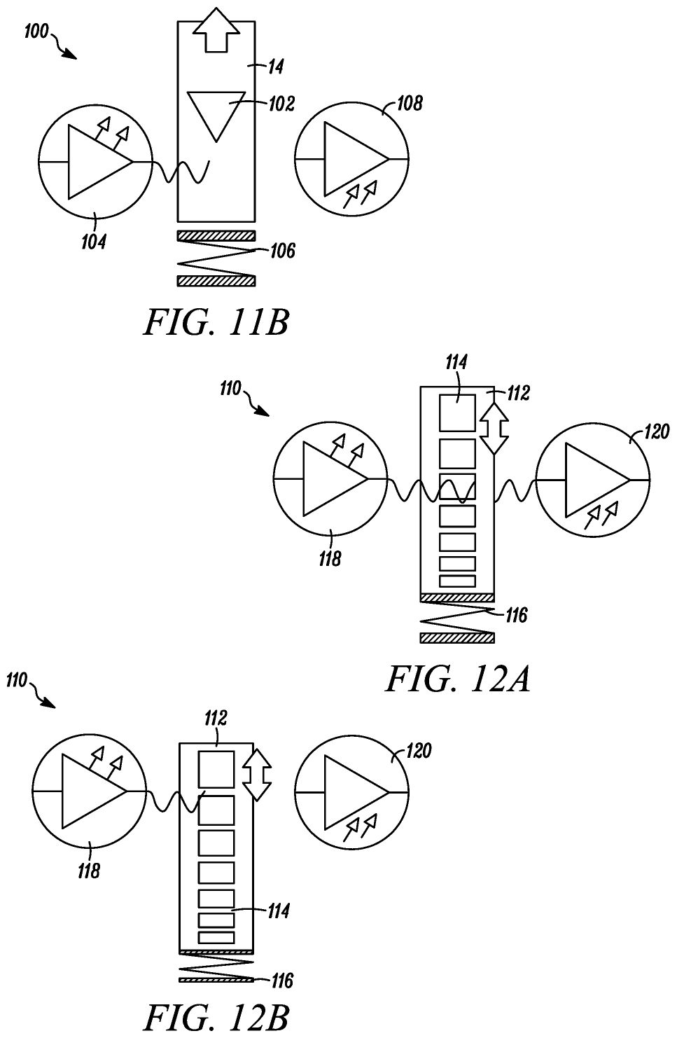

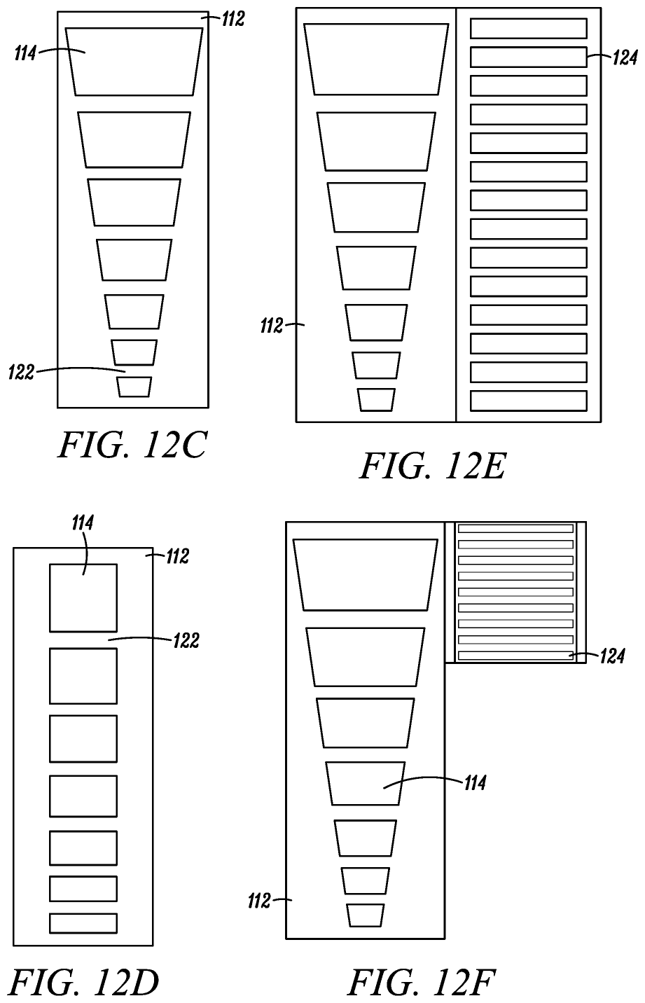

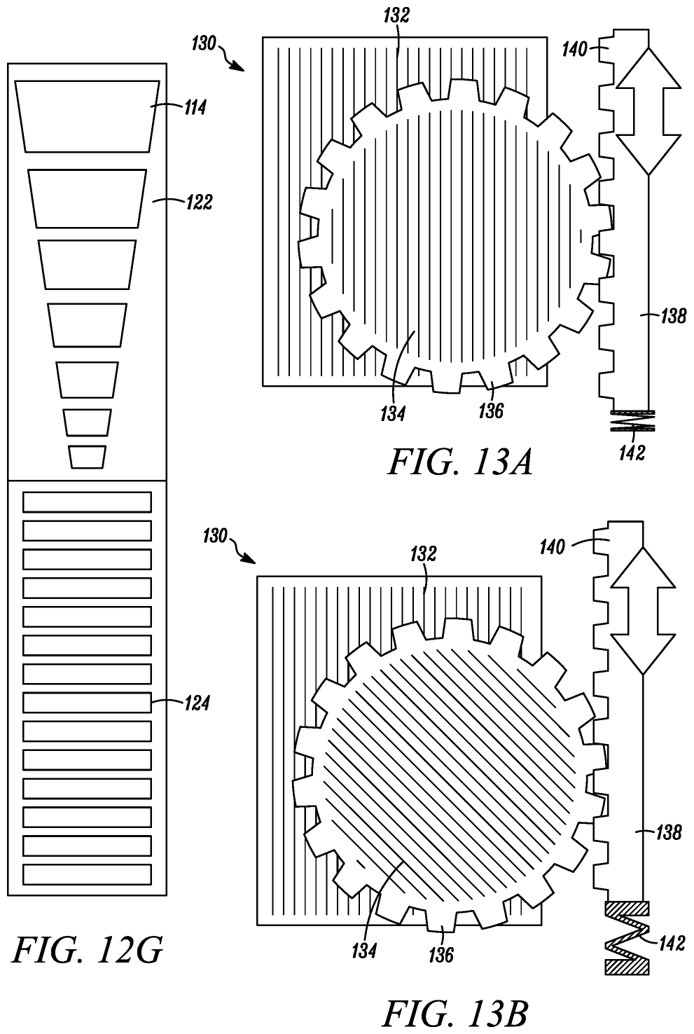

In addition to or alternative to one or more of the load sensor assemblies previously described, an optical encoder sensor assembly 110 can be used, illustrated in FIGS. 12A-12G. The optical encoder sensor assembly 110 includes a template 112 formed in the sensor body, or alternatively, directly formed in the leg 14 of the substrate 10. The template 112 has multiple openings 114, which can vary in height, or both height and width, as shown in FIG. 12C. The sensor, or leg 14, is positioned on a spring-like device 116 or a flexible substrate as previously described, that is sufficiently flexible to allow for movement of the template distances approximating the length of the template. A light 118 is positioned to shine though the template 112 and is positioned such that a base line “no presence” on the substrate 10 is known. The template 112 moves up and down due to forces on the substrate 10 such as weight and movement. The progressive variation in template opening sizes changes the amount of the light that passes through the template 112 and a photodiode 120 on an opposite side of the template 112 measures the amount of light. The dividers 122 between openings 114 in the template 112 provide reference points. The timing and frequency of the light passing through can be used to determined weight and movement of the subject.

The template 112 may be formed of fine, fixed size openings 114. The finer slits in the template 112 increases resolution of the light passing through, providing for more sensitive measurements. A combination of templates 112 may be used in the assembly 110 to provide both large signals and fine signals, illustrated in FIGS. 12E-12G. The fine-holed template 124 may require less area as the range of movement is much smaller, as shown in FIGS. 12E-12G. The templates 112 can be formed side by side and may only require one light source 118 and one photodiode 120, as in FIGS. 12E and 12F. The templates 112 can be formed one on top of the other as in FIG. 12G, with two separate light sources 118 and photodiodes 120 used. The large signals can provide information as to presence and weight thresholds or ranges. The large signals can also provide information as to movements such as turning over on the substrate. The fine-holed template 112 can be used to determine biometrics such as heartbeat and respiration.

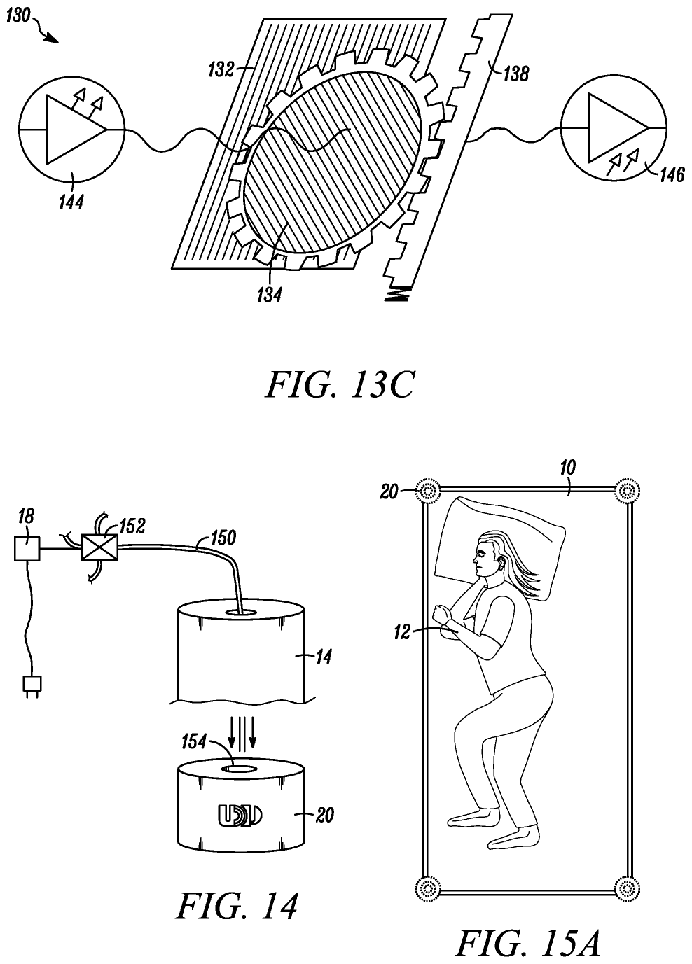

In addition to or alternative to one or more of the load sensor assemblies previously described, a polarized sensor assembly 130 can be used. The polarized sensor assembly 130 is illustrated in FIGS. 13A-C. The polarized sensor assembly 130 includes two polarized lenses, one being a stationary lens 132 and the other being a movable lens 134. The movable lens 134 is configured to be moved by a load on the substrate 10, the load transferred to the leg 14 and moving the movable lens 134. As a non-limiting example, the movable lens 134 can be a gear with teeth 136 along its perimeter. A sensor portion 138 positioned on the leg 14 of the substrate 10, or formed in the leg 14 of the substrate 10, also has teeth 140, with the teeth 136 of the movable lens 134 and the teeth 138 of the sensor portion 138 meshing together. The sensor, or leg, is positioned on a spring-like device 142 or a flexible substrate as previously described, that is sufficiently flexible to allow for movement of the sensor portion 138 to move the movable lens 134 between alignment and unalignment with the stationary lens 132. When a load is applied to the substrate 10, the sensor portion 138 moves, thereby moving the movable lens 134. A light 144 is transmitted to the lenses 132, 134, and when the polarized lenses are aligned as in FIG. 13A, the light is transmitted through the lenses 132, 134. When the polarized lenses are unaligned to different degrees, the light is filtered to different degrees. The light 144 transmitted through the lenses is measured with a photodiode 146. The changes in light intensity can be used to measure minute movements that are then ran through the algorithms to determine data about the subject 12. For example, a base-line of no presence on the substrate may be set to complete alignment of the stationary and the movable lenses 132, 134. A weight threshold or ranges can be determined by an overall large movement of the movable lens 134, while minute changes in the light intensity and its frequency can determine respiration and heart rate. Moderate changes in light may indicate movement of the subject 12 on the substrate 10, such as moving a leg or arm.

One or more of any combination of the sensor assemblies described herein can be used in the systems herein. Each of the sensor assemblies can be powered with any means known to those skilled in the art. Conventional electrical power may be used to power the sensor assemblies, or each sensor assembly may have a battery. In one example shown in FIG. 14, power can be delivered to the sensor assemblies 20 via a fiber optic cable 150. The fiber 150 can be run down the leg 14 to the sensor assembly 20. Light 152 from the fiber 150 is converted to power via a solar cell or photodiode 154 located at the sensor assembly 20 location. Data transmission to the local controller 18 or processor can be wired or wireless. The same fiber optic cable 150 can be used to transfer data from the sensor assemblies 20 to a processor as an alternative to, or in addition to, BLE or Wifi. One color (wavelength) of light can be used for power and a second color (wavelength) can be used for data transfer.

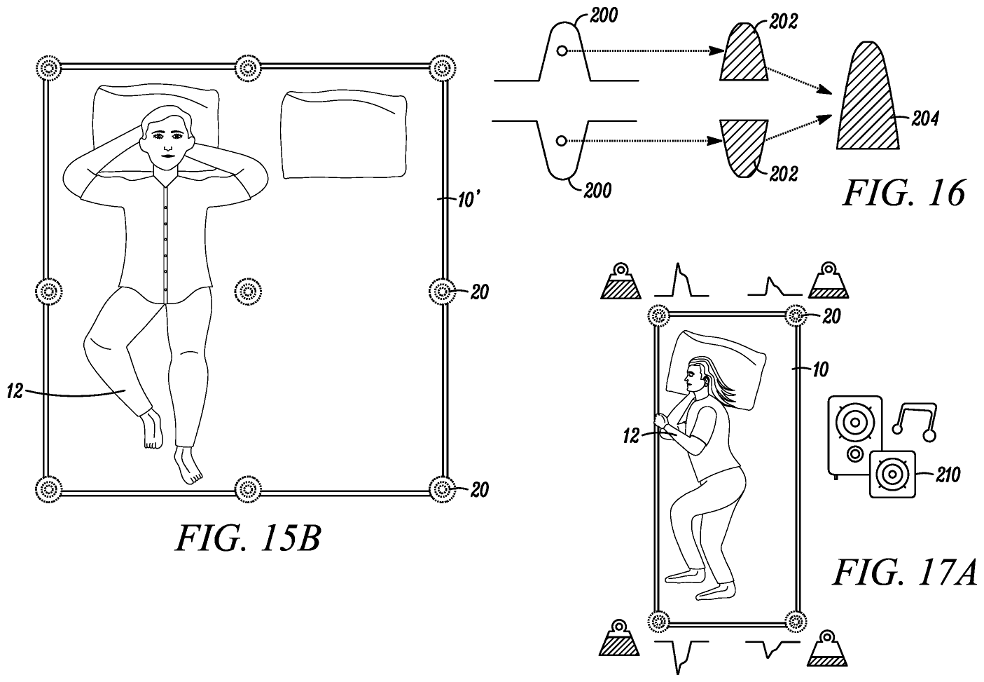

An example of a configuration of the load sensor assemblies 20 for use with a substrate 10 on which one subject 12 is designed to rest is illustrated in FIG. 15A. Four sensor assemblies 20 are positioned at the legs 14 in the four corners of the substrate 10. Although four sensor assemblies 20 are illustrated, the system would automatically reconfigure for more or less sensor assemblies 20. However, a load sensor assembly 20 is required at each location in which a load is transferred from the substrate 10 to the floor 16. For a substrate 10′ on which two people are designed to rest, nine sensor assemblies 20 may be used, as illustrated in FIG. 15B. Although nine sensor assemblies 20 are illustrated, the system 1 would automatically reconfigure for more or less sensor assemblies. For example, for beds in which two twins are placed together, eight sensor assemblies 20 may be used, one for each of the four legs of the two twin beds. Using a system 1 with multiple sensor assemblies 20 provides the ability to remove or cancel out or combine signals from another subject or the environment. The signals from multiple sensors are combined and/or separated to enhance the amplitude, reduce noise and increase the usefulness of various biometrics. The use of multiple sensors in a substrate on which two people rest provides the ability to map and correlate each person's weight, position and bio signals while they are on the subject at the same time. The system can also distinguish between the people when they are on the substrate alone.

Examples of data determinations that can be made using the systems herein are described. The algorithms are dependent on the number of sensors and each sensor's angle and distance with respect to the other sensors. This information is predetermined. Software algorithms will automatically and continuously maintain “empty weight” calibration with the sensors so that any changing in weight due to changes in a mattress or bedding is accounted for.

The load sensor assemblies herein utilize macro signals and micro signals and process those signals to determine a variety of data, described herein. Macro signals are low frequency signals and are used to determine weight and center of mass, for example. The strength of the macro signal is directly influence by the subject's proximity to each sensor.

Micro signals are also detected due to the heartbeat, respiration and to movement of blood throughout the body. Micro signals are higher frequency and can be more than 1000 times smaller than macro signals. The sensors detect the heart beating and can use this amplitude data to determine where on the substrate the heart is located, thereby assisting in determining in what direction and position the subject is laying. In addition, the heart pumps blood in such a way that it causes top to bottom changes in weight. There is approximately seven pounds of blood in a human subject, and the movement of the blood causes small changes in weight that can be detected by the sensors. These directional changes are detected by the sensors. The strength of the signal is directly influenced by the subject's proximity to the sensor. Respiration is also detected by the sensors. Respiration will be a different frequency than the heart beat and has different directional changes than those that occur with the flow of blood. Respiration can also be used to assist in determining the exact position and location of a subject on the substrate. These bio-signals of heart beat, respiration and directional movement of blood are used in combination with the macro signals to calculate a large amount of data about a subject, including the relative strength of the signal components from each of the sensors, enabling better isolation of a subject's bio-signal from noise and other subjects.

As a non-limiting example, the cardiac bio-signals in the torso area are out of phase with the signals in the leg regions. This allows the signals to be subtracted which almost eliminates common mode noise while allowing the bio-signals to be combined, increasing the signal to noise by as much as a factor of 3 db or 2× and lowering the common or external noise by a significant amount. By analyzing the phase differences in the 1 hz to 10 hz range (typically the heart beat range) the angular position of a person laying on the bed can be determined. By analyzing the phase differences in the 0 to 0.5 hz range, it can be determined if the person is supine or laying on their side, as non-limiting examples.

Because signal strength is still quite small, the signal strength can be increased to a level more conducive to analysis by adding or subtracting signals 200, resulting in larger signals. The signal deltas 202 are combined in signal 204 to increase the signal strength for higher resolution algorithmic analysis, as illustrated in FIG. 16.

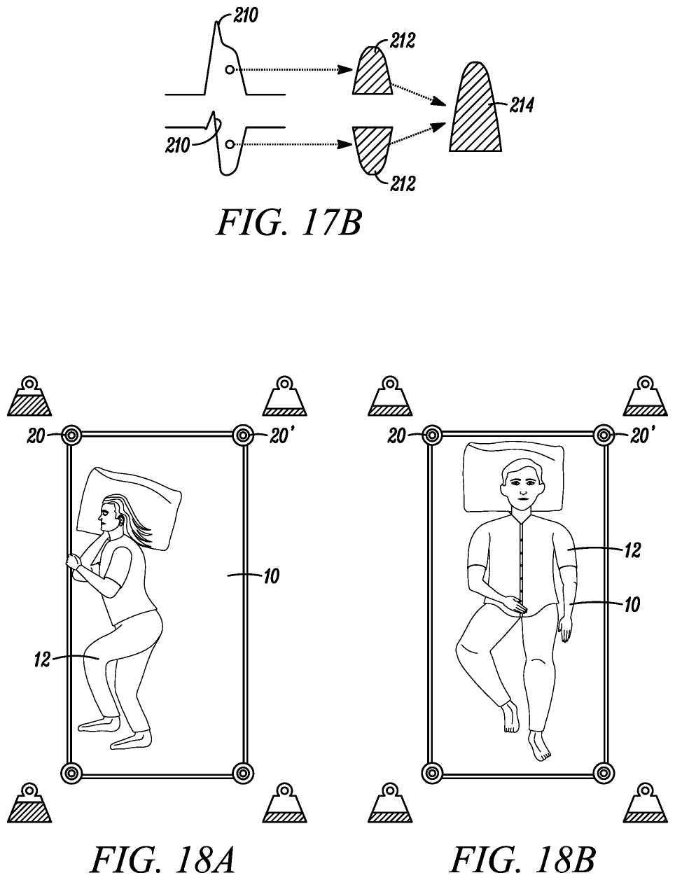

The systems 1 herein can cancel out external noise that is not associated with the substrate 10. External noise 210, such as the beat of a bass or the vibrations caused by an air conditioner, register as the same type of signal on all sensor assemblies 20 and is therefore canceled out when deltas are combined during processing. This is illustrated in FIGS. 17A and 17B. In FIG. 17B, the external noise 210 is shown on each signal 212, with the external noise removed and then the signals combined in 214.

Using superposition analysis, two subjects can be distinguished on one substrate. Superposition simplifies the analysis of the signal with multiple inputs. The usable signal equals the algebraic sum of the responses caused by each independent sensor acting alone. To ascertain the contribution of each individual source, all of the other sources first must be turned off, or set to zero. This procedure is followed for each source in turn, then the resultant responses are added to determine the true result. The resultant operation is the superposition of the various sources. By using signal strength and out-of-phase heart rates, individuals can be distinguished on the same substrate.

The systems 1 and sensor assemblies 20 herein provide the ability to provide dynamic center of mass location and movement vectors for the subject, while eliminating those from other subjects and inanimate objects or animals on the substrate. By leveraging multiple sensor assemblies that detect the z-axis of the force vector of gravity, and by discriminating and tracking the center of mass of multiple subjects as they enter and move on a substrate, not only can presence, motion and cardiac and respiratory signals for the subject be determined, but the signals of a single or multiple subjects on the substrate can be enhanced by applying the knowledge of location to the signal received. By analyzing the bio-signal's amplitude and phase in different frequency bands, the center of mass for a subject can be obtained using multiple methods, examples of which include:

DC weight;

AC low band analysis of signal, center of mass and back supine respiratory identification of subject;

AC mid band analysis of signal center of mass and cardiac identification of subject; and

AC upper mid band identification of snorer or apnea events.

The systems 1 and sensor assemblies 20 can be used to detect presence and location X, Y, theta, back and supine positions of a subject on a substrate. Such information is useful for calculating in/out statistics for a subject such as: period of time spent in bed, time when subject fell asleep, time when subject woke up, time spent on back, time spent on side, period of time spent out of bed. The sensor assemblies can be in sleep mode until the presence of a subject is detected on the substrate, waking up the system.

Macro weight measurements can be used to measure the actual static weight of the subject as well as determine changes in weight over time. Weight loss or weight gain can be closely tracked as weight and changes in weight can be measured the entire time a subject is in bed every night. This information may be used to track how different activities or foods affect a person's weight. For example, excessive water retention could be tied to a particular food. In a medical setting, for example, a two-pound weight gain in one night or a five-pound weight gain in one week could raise an alarm that the patient is experiencing congestive heart failure. Unexplained weight loss or weight gain can indicate many medical conditions. The tracking of such unexplained change in weight can alert professionals that something is wrong.

FIGS. 18A and 18B illustrate an example analysis of center of mass or position using macro signals. The load sensor assemblies 20 detecting the entire load on the substrate 10 triangulate a location of the center of mass by detecting weight measured by each load sensor assembly 20. In FIG. 18A, both load sensor assemblies 20 on the left side of the substrate 10 measure a similar weight that is greater than the weight measured by the load sensor assemblies 20′ on the right side of the substrate 10. The subject 12 is determined to be on the left side of the substrate 10. FIG. 18B illustrates the straight forward embodiment where the subject 12 is directly in the center of the substrate 10, based on each load sensor assembly 20 measuring the same weight.

Center of mass can be used to accurately heat and cool particular and limited space in a substrate 10, with the desired temperature tuned to the specific subject 12 associated with the center of mass, without affecting other subjects on the substrate 10. Certain mattresses are known to provide heating and/or cooling. As non-limiting examples, a subject can set the controller 18 to actuate the substrate to heat the portion of the substrate under the center of mass when the temperature of the room is below a certain temperature. The subject can set the controller 18 to instruct the substrate to cool the portion of the substrate under the center of mass when the temperature of the room is above a certain temperature.

These macro weight measurements can also be used to determine a movement vector of the subject. Subject motion can be determined and recorded as a trend to determine amount and type of motion during a sleep session. This can determine a general restlessness level as well as other medical conditions such as “restless leg syndrome” or seizures.

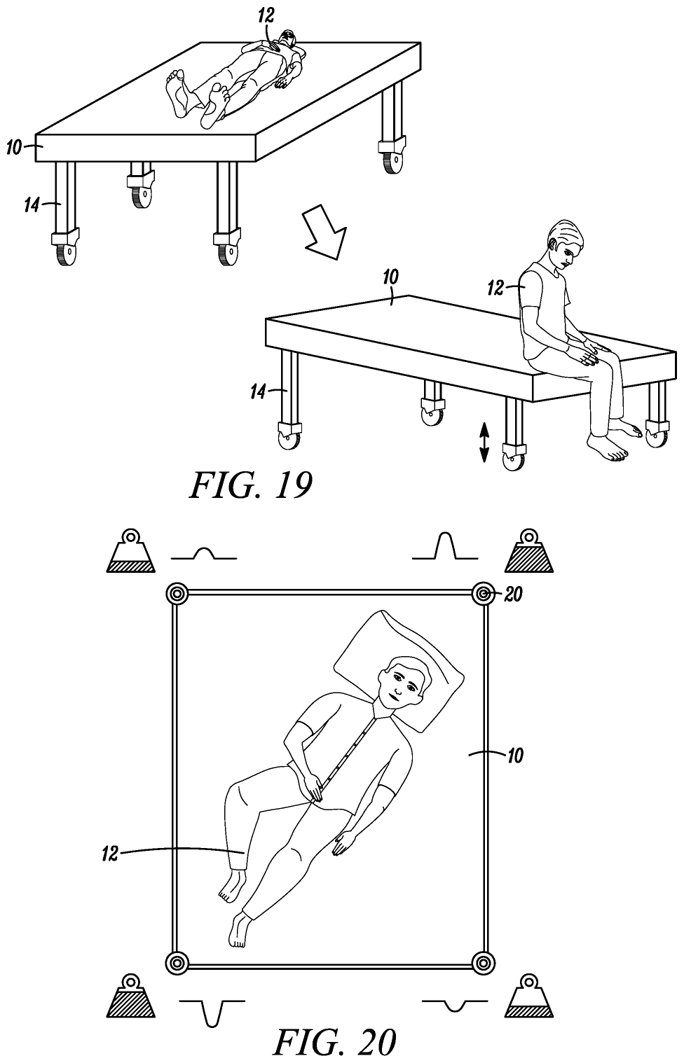

Motion detection can also be used to report in real time a subject exiting from the substrate. Predictive bed exit is also possible as the position on the substrate as the subject moves is accurately detected, so movement toward the edge of a substrate is detected in real time. In a hospital or elder care setting, predictive bed exit can be used to prevent falls during bed exit, for example. An alarm might sound so that a staff member can assist the subject exit the substrate safely. Alternatively, the legs 14 of the substrate 10 can be configured to lower on the side of the substrate 10 in which the subject 12 is exiting, so that the subject 12 can exit more easily. The legs 14 may be telescoping, for example, so that they increase and decrease in length. The legs 14 may be controlled by the controller 18 that receives the signals from the sensor assemblies 20 and processes the signals, sending programmed instructions to an actuator that lowers the legs 14 on the appropriate side, as illustrated in FIG. 19.

The systems 1 and sensor assemblies 20 can be used to determine actual positions of the subject on the substrate, such as whether the subject is on its back, side, or stomach, and whether the subject is aligned on the substrate vertically, horizontally, with his or her head at the foot of the substrate or head of the substrate, or at an angle across the substrate. The sensors can also detect changes in the positions, or lack thereof. In a medical setting, this can be useful to determine if a subject should be turned to avoid bed sores. In a home or medical setting, firmness of the substrate can be adjusted based on the position of the subject. For example, in FIG. 20, sleeping angle can be determined from center of mass, position of heart beat and/or respiration, and directional changes due to blood flow.

Controlling external devices such as lights, ambient temperature, music players, televisions, alarms, coffee makers, door locks and shades can be tied to presence, motion and time, for example. As one example, the controller 18 can collect signals from each load sensor assembly 20, determine if the subject is asleep or awake and control at least one external device based on whether the subject is asleep or awake. The determination of whether a subject is asleep or awake is made based on changes in respiration, heart rate and frequency and/or force of movement. As another example, the controller 18 can collect signals from each load sensor assembly 20, determine that the subject previously on the substrate has exited the substrate and change a status of the at least one external device in response to the determination. As another example, the controller 18 can collect signals from each load sensor assembly 20, determine that the subject has laid down on the substrate and change a status of the at least one external device in response to the determination.

A light can be automatically dimmed or turned off by instructions from the controller 18 to a controlled device when presence on the substrate is detected. Electronic shades can be automatically closed when presence on the substrate is detected. The light can automatically be turned on when bed exit motion is detected or no presence is detected. Electronic shades can be opened when motion indicating bed exit or no presence is detected. If a subject wants to wake up to natural light, shades can be programmed to open when movement is sensed indicating the subject has woken up. Waking up can be detected by increased movement, more rapid heartbeat, etc. Sleep music can automatically be turned on when presence is detected on the substrate. Predetermined wait times can be programmed into the controller 18, such that the lights are not turned off or the sleep music is not started for ten minutes after presence is detected, as non-limiting examples.

The controller 18 can be programmed to recognize patterns detected by the load sensor assemblies 20. The patterned signals may be in a certain frequency range that falls between the macro and the micro signals. For example, a subject may tap the substrate three times with his or her hand, creating a pattern. This pattern may indicate that the substrate would like the lights turned out. A pattern of four taps may indicate that the subject would like the shades closed, as non-limiting examples. Different patterns may result in different actions. The patterns may be associated with a location on the substrate. For example, three taps near the top right corner of the substrate can turn off lights while three taps near the base of the substrate may result in a portion of the substrate near the feet to be cooled. Patterns can be developed for medical facilities, in which a detected pattern may call a nurse.

While the figures all illustrate the use of the sensor assemblies with a bed as a substrate, it is contemplated that the sensor assemblies can be used with chairs such as desks, where a subject spends extended periods of time. A wheel chair can be equipped with the sensors to collect signals and provide valuable information about a patient. The sensors may be used in an automobile seat and may help to detect when a driver is falling asleep or his or her leg might go numb. Furthermore, the bed can be a baby's crib, a hospital bed, or any other kind of bed.

Implementations of controller 18 and/or controller 21 (and the algorithms, methods, instructions, etc., stored thereon and/or executed thereby) can be realized in hardware, software, or any combination thereof. The hardware can include, for example, computers, intellectual property (IP) cores, application-specific integrated circuits (ASICs), programmable logic arrays, optical processors, programmable logic controllers, microcode, microcontrollers, servers, microprocessors, digital signal processors or any other suitable circuit. In the claims, the term “controller” should be understood as encompassing any of the foregoing hardware, either singly or in combination.

Further, in one aspect, for example, controller 18 and/or controller 21 can be implemented using a general purpose computer or general purpose processor with a computer program that, when executed, carries out any of the respective methods, algorithms and/or instructions described herein. In addition or alternatively, for example, a special purpose computer/processor can be utilized which can contain other hardware for carrying out any of the methods, algorithms, or instructions described herein.

The word “example,” “aspect,” or “embodiment” is used herein to mean serving as an example, instance, or illustration. Any aspect or design described herein as using one or more of these words is not necessarily to be construed as preferred or advantageous over other aspects or designs. Rather, use of the word “example,” “aspect,” or “embodiment” is intended to present concepts in a concrete fashion. As used in this application, the term “or” is intended to mean an inclusive “or” rather than an exclusive “or”. That is, unless specified otherwise, or clear from context, “X includes A or B” is intended to mean any of the natural inclusive permutations. That is, if X includes A; X includes B; or X includes both A and B, then “X includes A or B” is satisfied under any of the foregoing instances. In addition, the articles “a” and “an” as used in this application and the appended claims should generally be construed to mean “one or more” unless specified otherwise or clear from context to be directed to a singular form.

While the disclosure has been described in connection with certain embodiments, it is to be understood that the disclosure is not to be limited to the disclosed embodiments but, on the contrary, is intended to cover various modifications and equivalent arrangements included within the scope of the appended claims, which scope is to be accorded the broadest interpretation so as to encompass all such modifications and equivalent structures as is permitted under the law.

What is claimed is:

1. A system for measuring data specific to a subject using gravity, the system comprising:
a substrate on which a subject lies, the substrate having multiple legs extending from the substrate to a floor to support the substrate;
load sensor assemblies, each load sensor assembly associated with a respective leg and comprising:
a cap configured to receive a load from the substrate;
a base configured to provide contact with the floor, the base and cap configured to fit together to maintain alignment of the cap to the base while allowing vertical movement of the cap;
a load cell between the base and the cap, one of the base and cap configured to translate the load to the load cell; and
a printed circuit board that processes and outputs data from the load cell, wherein a combination of all load sensor assemblies receive an entire load to which the substrate is subjected.

2. The system of claim 1, wherein each load sensor assembly is built into the respective leg.

3. The system of claim 2, wherein the cap has a perimeter sized and shaped to be identical to a perimeter of the respective leg, with the base fitting within the cap.

4. The system of claim 2, wherein each load sensor assembly is built into a top of the respective leg, the base formed by the top of the respective leg and the cap in contact with the substrate, each load sensor assembly configured to receive all load translated through the respective leg.

5. The system of claim 2, wherein each load sensor assembly is located in-line with an upper portion and a lower portion of the respective leg and configured to receive all load translated through the respective leg.

6. The system of claim 2, wherein each load sensor assembly is located at a bottom of the respective leg, the cap formed by the bottom of the leg and the base in contact with the floor, each load sensor assembly configured to receive all load translated through the respective leg.

7. The system of claim 1, wherein the cap has a single sidewall and the base has a double sidewall configured to receive the single sidewall of the cap, the double sidewall configured to restrain the cap from lateral movement while allowing movement in a vertical direction.

8. The system of claim 1, wherein each leg has a wheel and each load sensor assembly is located in the floor such that the cap is flush with the floor, each load sensor assembly spaced such that a load sensor assembly is under the respective leg of the substrate when the substrate is rolled into a use position.

9. The system of claim 1, further comprising a floor mat, wherein each load sensor assembly is located in the floor mat, the floor mat sized to have an area at least as large as an area of the substrate, each load sensor assembly positioned within the mat such that each load sensor assembly is under the respective leg of the substrate when the substrate is positioned on the mat.

10. The system of claim 1, wherein each load sensor assembly comprises multiple load cells positioned in the base, the cap configured with a circuit contact surface configured to translate the load equally to each of the multiple load cells.

11. The system of claim 1, further comprising a controller in communication with each load sensor assembly, the controller configured to collect signals from each load sensor assembly and determine a center of mass of the subject on the substrate.

12. The system of claim 1, further comprising a controller in communication with each load sensor assembly and at least one external device in communication with the controller, the controller configured to:
collect signals from each load sensor assembly;
determine if the subject is asleep or awake; and
control the at least one external device based on whether the subject is asleep or awake.

13. The system of claim 1, further comprising a controller in communication with each load sensor assembly and at least one external device in communication with the controller, the controller configured to:
collect signals from each load sensor assembly;
determine that the subject previously on the substrate has exited the substrate; and
change a status of the at least one external device in response to the determination.

14. The system of claim 1, further comprising a controller in communication with each load sensor assembly and at least one external device in communication with the controller, the controller configured to:
collect signals from each load sensor assembly;
determine that the subject has laid down on the substrate; and
change a status of the at least one external device in response to the determination.

15. A system for measuring data specific to a subject using gravity, the system comprising:
a substrate on which a subject rests, the substrate having multiple legs extending from the substrate to a floor to support the substrate;
at least two load sensor assemblies, each load sensor assembly associated with a respective leg configured to measure a static load and changes in load on the substrate through the leg;
a controller; and
communication means from each of the at least two load sensor assemblies to the controller, wherein the controller processes output from each of the at least two load sensor assemblies.

16. The system of claim 15, wherein each load sensor assembly comprises:
a cap configured to receive a load from the substrate;
a base configured to provide contact with the floor, the base and cap configured to fit together to maintain alignment of the cap to the base while allowing vertical movement of the cap;
a load cell between the base and the cap, one of the base and cap configured to translate the load to the load cell; and
a printed circuit board that processes and outputs data from the load cell to the processor.

17. The system of claim 16, wherein each load sensor assembly is built into the respective leg and the cap has a perimeter sized and shaped to be identical to a perimeter of the respective leg.

18. The system of claim 15, further comprising at least one external device in communication with the controller, the controller configured to:
collect signals from each load sensor assembly;
determine if the subject is asleep or awake; and
control the at least one external device based on whether the subject is asleep or awake.

19. The system of claim 1, further comprising at least one external device in communication with the controller, the controller configured to:
collect signals from each load sensor assembly;
determine that the subject previously on the substrate has exited the substrate; and
change a status of the at least one external device in response to the determination.

20. The system of claim 1, further comprising at least one external device in communication with the controller, the controller configured to:
collect signals from each load sensor assembly;
determine that the subject has laid down on the substrate; and
change a status of the at least one external device in response to the determination.
